(19) United States
(12) Patent Application Publication
(10) Pub. No.: US 2026/0258350 A1
(43) Pub. Date: Sep. 3, 2026

(54) 3D Bioprinted Model for Quantifying Neurite Outgrowth

(71) Applicant: Merck Sharp & Dohme LLC, Rahway, NJ (US)

(72) Inventors: Chloe Ann Whitehouse, London (GB); Yufang He, Milltown, NJ (US); Nicola Corbett, London (GB)

(21) Appl. No.: 19/553,137

(22) Filed: Feb. 27, 2026

Related U.S. Application Data
(60) Provisional application No. 63/765,362, filed on Feb. 28, 2025.

(51) Int. Cl. C12N 5/0793; B33Y 80/00; C12M 1/34; C12N 5/0797

(52) U.S. Cl. CPC C12N 5/0619; B33Y 80/00; C12M 41/46; C12N 5/0623; C12N 2503/02; C12N 2506/45; C12N 2513/00; C12N 2533/52; C12N 2533/80

(57) ABSTRACT

A three-dimensional (3D) bioprinted model of a forebrain cortex is designed to quantify neurite outgrowth across a hydrogel bridge. The 3D bioprinted model includes two cell-laden compartments formed using a first hydrogel matrix, separated by a hydrogel bridge formed from a second, stiffer hydrogel matrix. The model may fit within standard 96-well plate formats, enabling medium-throughput screening applications. The model was validated using Alzheimer's disease forebrain cortical populations, demonstrating significant reductions in neurite outgrowth from Alzheimer's disease populations compared to controls.

### CROSS-REFERENCE TO RELATED APPLICATIONS

This Application clams the benefit of U.S. Provisional Patent Application No. 63/765,362, filed Feb. 8, 2025, which is incorporated by reference.

### BACKGROUND

### 1. Technical Field

The subject matter described relates generally to 3D bioprinted structures and, in particular, to 3D bioprinted structures that model neurite outgrowth in the forebrain cortex of a brain.

### 2. Problem

The study of neurological diseases, including Alzheimer's disease (AD), has been limited by a reliance on two-dimensional (2D) in vitro models, as these models can fail to replicate the complex pathological changes and intricate three-dimensional (3D) architecture of the human brain. AD is often characterized by amyloid-β (Aβ) and tau dysfunction, however, progressive synaptic loss and neurite degeneration, alongside changes to mitochondrial function, oxidative stress, and proteostasis, are also associated with disease progression. These pathological hallmarks ultimately lead to memory deficits and cognitive decline.

Mitochondrial dysfunction and increased oxidative stress are well-recognized contributors to neurite degeneration in AD, as mitochondria are critical for ATP production, calcium buffering, and energy supply along extending neurites. Swedish and Indiana APP mutations have been widely suggested to result in mitochondrial dysfunction and increased production of reactive oxygen species (ROS). Poor processing of APP and the resultant changes to amyloid processing pathways causes mitochondrial stress and damage to mitochondrial DNA, driving cellular energy imbalances. The dysfunctional mitochondria are not broken down by mitophagy, and this results in oxidative stress. APP C-terminal fragments have been implicated in mitochondrial dysfunction and proteostasis deficits.

While 2D cell culture systems have provided valuable insights into AD, they lack the true spatial, mechanical, and biochemical cues that are necessary to fully recapitulate cell-cell interactions and network-level pathologies. Alongside traditional 2D culture systems, transgenic rodent models have also been instrumental in studying AD. In vivo models benefit from the ability to track disease progression over time and correlate underlying pathologies with behavioral studies. The broad availability of different transgenic models allows for investigation of both Aβ and tau pathology within the context of a functional brain, and the interactions of all cell types (including the blood-brain barrier and microglia) alongside the presence of biochemical and biomechanical cues, allows deep understanding of the disease pathology. In contrast, advanced 3D in vitro models offer a controlled and human-relevant microenvironment, and while they may lack whole-organism interactions, these models can excel in their practical advantages, including cost-effectiveness, scalability, and compatibility with high-content assay formats. Additionally, the use of animal models presents ethical concerns, and efforts should be made to refine, reduce, and replace the use of these model systems within all research studies, paving the way for a new generation of complex in vitro models. Consequently, there is a growing demand for advanced 3D culture systems which can capture the intricacies of neuronal maturation, synaptic connectivity, and pathological cellular stress responses.

Existing 3D neural models, including organoids and neurospheroids, offer considerable advantages over traditional 2D cultures, such as enhanced neuronal differentiation, maturation, and network formation. However, these systems often face technical challenges that limit their implementation in quantitative assays. Neural organoids, while physiologically relevant, are time-intensive to generate, lack reproducibility, and are challenging to image due to their dense cellular architecture. Neurospheroids and assembloids, while faster to produce, similarly suffer from imaging limitations which hinder accurate quantification of neurite growth parameters.

### SUMMARY

The above and other problems may be addressed by a novel tri-matrix 3D bioprinted model of the human forebrain cortex, containing glutamatergic neurons, GABAergic neurons, astrocytes, and microglia, to enable real-time quantification of neurite outgrowth and synaptic connectivity. In various embodiments, this platform incorporates a hydrogel bridge within a scaffold-based 3D bioprinted construct, providing a controlled and optically clear area within the model for monitoring the growth of neural projections over time. By selecting an appropriate hydrogel bridge stiffness to slow cell migration across matrices, the system can facilitate high-resolution imaging while maintaining cell viability and promoting neuronal differentiation. A 96-well plate format may be used to support medium-throughput applications, enabling robust and reproducible quantification of neurite outgrowth metrics across multiple replicates. This advancement addresses limitations in current 3D culture systems, enabling its use as a screening platform for therapeutics targeting neurite regeneration and connectivity deficits.

### BRIEF DESCRIPTION OF THE DRAWINGS

FIG. 1 illustrates the production and application of 3D bioprinted forebrain models to evaluate synapse formation and other activity of neurites, according to one embodiment.

FIG. 2 illustrates 3D bioprinted model design and development, according to one embodiment. (A) Culture timeline of 3D model generation from iPSC stage to assay (Day 51-53) (B) Brightfield images (10×) of NPCs in matrix selection print run, table corresponding table shows matrix name and key composition. (C) 3D bioprinted model structure and dimensions, empty 96-well is indicated in orange, hydrogel matrix in purple, and cell clusters in green and pink. (D) Brightfield images of neuron differentiation and connectivity in 3D imaging model between Day 7 and Day 25, white arrows highlight areas of neurite outgrowth. Scale bars represent 200 μm.

FIG. 3 shows cell types present in forebrain cortical models, according to one embodiment. (A) Neuronal marker (βIII-tubulin, fuchsia), astrocyte marker (S100β, green), and microglia marker (IBA1, yellow) expression in 2D and 3D culture conditions, with nuclear marker (DAPI, blue). Scale bars represent 100 μm. (B) Compiled Z-stack exhibiting morphology and expression of neuronal marker (βIII-tubulin, fuchsia), astrocyte marker (S100β, green) in 3D models, with nuclear marker (DAPI, blue). Scale bars represent 10 μm and 20 μm (left to right). (C) Ratio of astrocytic (S100β+) cells to neuronal cells (βIII-tubulin+) in 2D and 3D culture conditions were averaged across 12 models per bioprint, for a total of 3 bioprints per condition (mean±SEM). (D) Compiled Z-stack and surface render of microglial cells (IBA, green) in 3D culture conditions, with nuclear marker (DAPI, blue). Scale bars represent 30 μm and 20 μm (left to right).

FIG. 4 shows neural connectivity in a 3D model, according to one embodimet. (A) Neuronal marker (MAP2, red) and nuclear marker (DAPI, blue) shows neurite extensions within 3D hydrogel matrix, first column shows individual imaging planes, second column shows compiled Z-stack, third column shows filament tracing performed in Imaris software. Scale bars represent 100 μm, 50 μm and 20 μm (left to right). (B) Whole well scan of large plug model with live plasma membrane stain (Cell Mask, green) on Day 6 and Day 15 post-bioprinting, and high-magnification images of neurite outgrowth in same model once fixed and stained for neuronal marker (βIII-tubulin, green) and nuclear marker (DAPI, blue). Scale bars represent 400 μm, 400 μm, 20 μm and 50 μm (left to right).

FIG. 5 shows connectivity and synaptic protein expression in 3D AD models, according to one embodiment. (A) 3D model morphology in healthy isogenic control models and AD mutant models at Day 25 post-bioprinting using live cell plasma membrane dye (Cell Mask, green). Scale bars represent 400 μm (B) Expression of fluorescent mKate2 synapsin protein (red) in same models as [A] following transfection with Neurolight lentivirus. Scale bars represent 400 μm (C) Filament traces of neurite outgrowths (yellow) in hydrogel bridge rendered in Imaris software from βIII-tubulin staining in healthy isogenic controls and AD (APP) mutant models. Each image represents a random field from a model within 1 bioprint. Scale bars represent 50 μm (D) Neurites per soma values qualitatively extracted from confocal images taken from 3 models of 1 bioprint. (E) Synapsin-1 expression as quantified from synapsin-1 immunostaining high content imaging; results were averaged across 12 models per bioprint, for a total of 3 bioprints per condition, shown as mean±SEM with significance indicated as per analyzed using 2-way ANOVA. (F) Compiled Z-stack images of Synapsin-1 stain (fuchsia) in 3D AD models (APP) and healthy isogenic controls. Scale bars represent 40 μm, 20 μm and 20 μm (left to right).

FIG. 6 shows Amyloid-β dysregulation in 3D models, according to one embodiment. (A) Immunostaining of APP C-Terminal fragment (CTF, green) with neuronal marker (βIII-tubulin, fuchsia) and nuclear marker (DAPI, blue) in AD mutants and isogenic controls in 3D. White arrows indicate example areas of APP staining, final column shows surface render of APP staining separate to neuronal stain. Scale bars represent 2 μm, 10 μm and 30 μm (left to right). (B) Aβ isoform quantification for Aβ40 and Aβ42, and the ratio of the two Aβ isoforms, in 2D and 3D AD and isogenic control samples. Shown as normalized to total protein in sample & averaged across 12 models per bioprint, for a total of 3 bioprints per condition, statistical significance is indicated as per analysis with 2-way ANOVA (mean±SEM). (C) Immunostaining of amyloid-β protein (6E10, orange) and neuronal marker (βIII-tubulin) with nuclear stain (DAPI, blue) in AD mutant and healthy isogenic control 3D models. White arrows indicate areas of amyloid-β accumulation in hydrogel. Scale bars represent 50 μm.

FIG. 7 shows cellular pathologies in AD 3D models, according to one embodiment. (A) Quantification of live TMRM stain intensity as a measure of mitochondrial health in AD mutant and isogenic control 2D and 3D models, normalized to area of live cells. (B) Quantification of TOMM20 immunostaining in AD mutant and isogenic control 2D and 3D models, normalised to nuclei stained with DAPI. (C) Quantification of phospho-Ubiquitin immunostaining in AD mutant and isogenic control 2D and 3D models, normalized to nuclei stained with DAPI. For [A-C] results were averaged across 12 models per bioprint, for a total of 3 bioprints per condition, significance is shown as per analysis with 2-way ANOVA, (mean±SEM). (D) Immunostaining of mitochondrial marker (TOMM20, fuchsia) and nu-clear stain (DAPI, blue), shown as individual plane (column one), Z-stack (column two), and surface render using Imaris (column three), in AD mutant and isogenic control 3D models (1 model per image within 1 bioprint). White arrows show areas of altered mitochondrial morphology. Scale bars represent 10 μm, 5 μm and 5 μm (left to right). (E) Qualitative analysis of image parameter “TOMM 20+objects” from surface render images from 3 models of 1 bioprint (mean±SEM). (F) Qualitative analysis of image parameter “TOMM20+total volume” from surface render images from 3 models of 1 bioprint (mean±SEM). (G) Quantification of live DHE stain intensity as a measure of ROS production in AD mutant and isogenic control 2D and 3D models, normalized to area of live cells, averaged across 12 models per bioprint, for a total of 3 bioprints per condition (mean±SEM) (H) Quantification of conditioned media GSH assay in AD mutant and isogenic control 2D and 3D models, normalized to total protein of samples and averaged across 24 models per bioprint, for a total of 3 bioprints per condition (mean±SEM).

FIG. 8 shows transcriptomic analysis of AD 2D and 3D models, according to one embodiment. (A) Bulk RNA-Seq analysis of isogenic control and AD mu-tant 2D and 3D models represented as sample quality control heatmap of Pearson correlation (B-G) Over representation analysis lollipop plots were produced from top ten enriched terms for each condition and direction, where each graph represents (B) upregulated pathways between isogenic control cells in 3D vs 2D conditions. (C) downregulated pathways between isogenic control cells in 3D vs 2D conditions. (D) upregulated pathways between isogenic control cells and AD mutant cells in 2D culture conditions. (E) downregulated pathways between isogenic control cells and AD mutant cells in 2D culture conditions. (F) upregulated pathways between isogenic control cells and AD mutant cells in 3D model conditions. (G) downregulated pathways between isogenic control cells and AD mutant cells in 3D model conditions.

### DETAILED DESCRIPTION

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### Overview

A novel 3D bioprinted model of the forebrain cortex is designed to quantify neurite outgrowth across a hydrogel bridge. The tri-matrix 3D bioprinted model comprises three distinct hydrogel regions with specific dimensional characteristics designed to facilitate controlled neurite outgrowth analysis. Specifically, the 3D bioprinted model has a pair of cell-laden compartments separated by a hydrogel bridge. In one embodiment, the cell-laden compartments are formed from a first hydrogel matrix with a first stiffness and the hydrogel bridge is formed from a second hydrogel having a second stiffness, greater than the first stiffness. Neural cells are embedded in the first hydrogel in the cell-laden compartments and second hydrogel provides an optically clear region in which neurite extension from the cell-laden compartments can be monitored. In some embodiments, the model is configured to fit within standard 96-well plate formats, enabling medium-throughput screening applications. Two primary model configurations have been developed to serve different experimental purposes: a large plug model and an imaging model.

The large plug model is designed for applications requiring substantial biomass, such as RNA extraction, protein analysis, and biochemical assays. In one embodiment, this configuration spans the full width of a standard 96-well plate well, having a diameter of approximately 10.5 mm. The large plug model may have a Z-height of 500 μm, providing sufficient three-dimensional volume to support robust cell populations. The two cell-laden compartments are formed using a first hydrogel matrix (e.g., a Cx02.99161 hydrogel matrix from Inventia Life Science), each occupying lateral portions of the well. These compartments are separated by the hydrogel bridge formed from a second hydrogel matrix (e.g., the Px03.36 hydrogel matrix from Inventia Life Science). The second hydrogel matrix may be stiffer than the first hygrogel matrix. In one embodiment, the bridge region is designed to be approximately 3.5 mm thick, creating an optically clear zone for monitoring neurite extension between the two cell populations.

The imaging model is specifically optimized for high-resolution confocal microscopy and live-cell imaging applications. In one embodiment, this smaller configuration has overall dimensions of approximately 2.2 mm in width and approximately 200 μm in Z-height. The reduced dimensions facilitate superior optical penetration and resolution, enabling detailed visualization of individual neurite processes, synaptic structures, and cellular morphology. The hydrogel bridge in the imaging model maintains similar compositional properties to the large plug model but is proportionally scaled to match the overall reduced dimensions. For example, the bridge width in the imaging model may be approximately 1.5 mm thick, providing adequate separation between cell populations while maintaining optical clarity for tracking neurite outgrowth dynamics.

For both imaging models, the hydrogel bridge serves as the functional element for neurite outgrowth quantification. In one embodiment, the increased stiffness (e.g., 3 kPa) of the hydrogel bridge relative to the stiffness (e.g., 1.1 kPa) of the cell-laden compartments is achieved through higher crosslinking density. The increased stiffness restricts migration of cell soma, which typically measure 8-15 μm in diameter, while permitting penetration of smaller neurite processes that range from 0.1-2 μm in diameter. The bridge maintains optical transparency, enabling continuous monitoring of neurite extension throughout the culture period.

To validate the models, Alzheimer's disease (AD) forebrain cortical populations derived from human iPSCs carrying APP (amyloid precursor protein) mutations (K670M/N671L+V717F) were cultured. The Swedish (K670M/N671L) and Indiana (V717F) mutations in APP promote increased Aβ production, particularly the aggregation-prone isoform of Aβ42, which drives pathology in familial AD. Swedish and Indiana APP mutations are able to disrupt neuronal growth, synaptic function, and mitochondrial health in vitro and in vivo. 3D culture systems have demonstrated the ability to amplify pathological phenotypes across many diseases, likely due to increased complexity in mimicking biomechanical properties of cell-cell interactions, and pathological changes to the extracellular matrix.

Neurite and synapse formation were significantly impaired in 3D AD mutant cultures compared to controls, but this was not replicated in 2D, highlighting deficits in these traditional 2D cell culture models. To investigate the mechanisms underlying impaired neurite outgrowth in 3D and 2D models of AD, amyloid-β dysfunction, mitochondrial health, and oxidative stress were assessed in both conditions. In the 3D model, APP mutant cultures exhibited reduced mitochondrial membrane potential and fragmented networks, indicating dysfunction and potential cellular energy deficits. The 3D APP mutant forebrain cortical populations exhibited fragmented, shorter neurites with reduced branching complexity compared to isogenic controls. Notably, these phenotypes were absent or significantly attenuated in APP mutant cultures grown under 2D conditions, highlighting the importance of the 3D scaffold for capturing these disease-relevant mechanisms. These findings align with clinical observations of synaptic loss and neurite degeneration in AD patients.

In addition to the ability to model neurite out-growth, the tri-matrix 3D bioprinted model provides a platform for other assay formats, including live assays, protein extraction, and cell retrieval from the scaffold. We use these quantitative measurements to investigate underlying cellular pathologies in the AD models which may contribute to the observed neurite outgrowth and connectivity changes. Mitochondrial dysfunction and increased oxidative stress are well-recognized contributors to neurite degeneration, as mitochondria are critical for ATP production, calcium buffering, and energy supply along extending neurites.

Additionally, elevated oxidative stress and proteostasis disruption were identified in the 3D AD models as indicators of cellular damage which may be limiting neurite extension. Furthermore, transcriptomic (bulk RNA-Seq) analysis revealed distinct differences in gene expression pathways between 2D and 3D models of AD, suggesting alternate underlying mechanisms of disease pathology between the culture conditions. These pathological features were more pronounced in 3D cultures compared to 2D, likely due to the increased energy demands and spatial constraints of the 3D environment. This demonstrates the functionality of the novel 3D bioprinted model for quantifying neurite connectivity and in identifying underlying disease mechanisms.

Taken together, the novel 3D bioprinted model enables real-time, high-resolution quantification of neurite outgrowth and connectivity that is scalable to at least medium throughout applications. The 3D model of AD has the ability to recapitulate pathological features, including impaired neurite growth, mitochondrial dysfunction, and cellular stress, which are not observed in 2D systems. The scalability and compatibility of the model with automated imaging technologies make it a promising tool for drug screening and mechanistic studies of neurodegenerative diseases. The platform can also be extended to other neurological disorders characterized by connectivity deficits, such as Parkinson's disease, traumatic brain injury, and neurodevelopmental disorders, further underscoring its versatility and translational potential.

### Example Materials and Methods

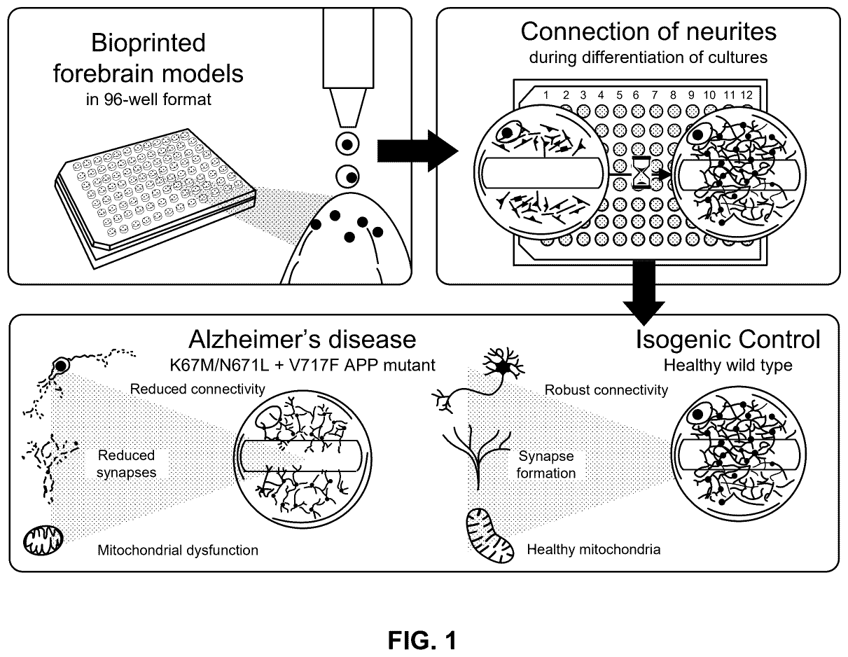

FIG. 1 illustrates one embodiment of an overall approach for producing and applying 3D bioprinted forebrain models for evaluating synapse formation and other activity of neurites. The forebrain models are 3D bioprinted in a 96-well plate (or another plate format). Each well in the plate includes a model with three portions. Two of the portions include neurites and are separated by a third portion, which is a band initially including no neurites. Over time, the neurites of a healthy, wildtype expand into the separating band and form synapse connections with other neurites. Conversely, neurites with the mutation associated with AD exhibit less growth into the band and form less synapses. Similarly, the wildtype neurites exhibit healthy mitochondria while the AD mutation neurites exhibit mitochondrial dysfunction.

The human healthy control iPSC line (XCL-1) was CRISPR-edited by Sigma-Aldrich to include Indiana and Swedish (KM670/671NL+V717F HOMO) APP mutations. Both healthy and APP mutant iPSCs were plated on 10 μg/mL matrigel (Gibco) and were maintained in mTESR plus (STEMCell Technologies), cells were passaged every 5-6 days using ReLeSR (STEMCell Technologies). ROCK inhibitor Y27632 (10 μM) was included in culture media for 24 hours after passaging to improve cell survival. Human iPSCs from the same line (XCL-1) were differentiated into induced microglia like-cells (iMGLs). All iPSC lines were procured and assessed by internal stem cell approval committees to verify ethical procurement of cells with appropriate informed patient consent documentation.

The generation of neural progenitor cells (NPCs) from iPSCs was performed using the SMADi Neural Induction Kit from STEMCell Technologies using the Monolayer Culture Protocol as per manufacturer protocols. Briefly, iPSCs were dissociated using Gentle Cell Dissociation Reagent at 37° C. for 8-10 minutes, and cell aggregates were broken up by pipetting the suspension 3-5 times using a 1 mL pipettor. Cells were added to a 15 mL falcon containing DMEM/F12 and were centrifuged for 300×g for 5 minutes. Cells were resuspended at a final concentration of 2×10⁵ cells/cm² in Neural Induction Medium with SMADi supplement on 6-well plates coated with Geltrex (Gibco) (1 hour at 37° C.). ROCK inhibitor Y27632 (10 μM) was included in culture media for 24 hours to improve cell survival. Daily media changes were performed until Day 7, when cells were passaged using Accutase (Gibco) and replated on Geltrex-coated 6-well plates at a concentration of 2×10⁵ cells/cm² in Neural Induction Medium with SMADi supplement. This process was repeated when cells were sufficiently expanded for passage on Day 14, and daily media changes were continued until Day 21. At Day 21, NPCs were fully differentiated and were passaged into Geltrex coated 6-well plates at a concentration of 1.25×10⁵ cells/cm² in Neural Progenitor Medium (STEMCell Technologies). After Day 21, Neural Progenitor Medium was changed 3 times per week. Cells were used for 3D bioprinting or 2D differentiation between Day 28 and Day 35, and NPCs were not used beyond P4. Cells from each batch were evaluated for expression of NPC markers using immunostaining for SOX1 and SOX2 before subsequent differentiation.

NPCs were maintained and expanded in Neural Progenitor Medium (STEMCell Technologies) until differentiation. Forebrain Neuron Differentiation and Maturation kits from STEMCell Technologies were used to differentiate NPCs as per manufacturer instructions. Briefly, NPCs were passaged using Accutase as described in previous section and plated in 96-well plates at a concentration of 0.8×10⁵ cells/cm² . After 3-5 days when cells had reached 80% confluence, media was changed from Neural Progenitor Medium into Forebrain Neuron Differentiation Medium. Daily full media changes of 200 μL Forebrain Neuron Differentiation Medium were performed for 5 days. On Day 6 of forebrain differentiation, media was changed to 200 μL Forebrain Neuron Maturation medium without passage. Cells were maintained in Forebrain Neuron Maturation medium for a further 15 days with half media changes (100 μL) three times per week. After 15 days in Forebrain Neuron Maturation medium, media was changed to iMGL maintenance medium: Advanced DMEM/F 12 without Phenol Red, 1×B27 supplement (Gibco), 1×N2 supplement (Gibco), 1×non-essential amino acids (NEAA) (Gibco), 1×GlutaMAX (Gibco), 2×ITSG solution (Gibco), 400 μM monothioglycerol (Sigma-Aldrich), 5 μg/mL human insulin (Gibco), 25 ng/mL m-CSF (preprotech) and 20 ng/mL IL-34 (preprotech). iMGLs were thawed and a final concentration of 5% of total cells were added to the forebrain population cultures. Forebrain cultures with iMGLs were maintained for 48 hours before assay. 3D bioprints were created using the RASTRUM™ bioprinter. 24 hours prior to bioprinting, ROCK inhibitor Y27632 (10 μM) was included in NPC culture media to improve cell survival during the print process. The model was designed using the RASTUM™ cloud, with multimatrix conformations “Triple Matrix-Large Plug” with custom spacing used for the large model, and the “Triple Matrix-Imaging Model” used for the imaging model. Three hydrogel matrices were trialed for ability to facilitate NPC expansion and differentiation: Px03.36 (3 kPa stiffness+RGD, IKVAV, YIGSR, with full length hyaluronic acid protein), Px02.36 (1.1 kPa stiffness+RGD, IKVAV, YIGSR, with full length hyaluronic acid protein), and Cx02.99161 (1.1 kPa stiffness+RGD, IKVAV, YIGSR, with full length hyaluronic acid protein and laminin-211). To create a model which could facilitate NPC expansion and differentiation in outer compartments, with later neurite growth into the hydrogel bridge, the following matrices were selected: Px03.36 for the hydrogel bridge and Cx02.99161 for the cell-laden matrices. Models were bioprinted into 96-well plates, and plate maps for each experiment were created on the RASTRUM™ cloud software. Wells used for 2D control experiments were pre-coated with Geltrex (1 hour at 37° C.) which were washed and replaced with 200 μL Neural Progenitor Medium before commencing the print process. Immediately before bioprinting, bioink fluids were thawed at room temperature and the printer was greenlighted according to manufacturer instructions. Once thawed, bio-ink fluids were added to the RASTRUM™ cartridge for inert base bioprinting into the wells of the 96-well plate which would contain 3D structures. During inert base printing, Accutase was added to NPC cultures to dissociate cells into a single cell suspension. Following dissociation, trypan blue was added, and live cells were counted. A total of 3.2×10⁶ NPCs were used per 200 μL of print activator fluid, total volume of activator fluid is determined by the RASTRUM software following print design. Total NPCs were centrifuged for 300×g for 5 minutes and were re-suspended in the total required volume of print activator fluid. After inert base printing was complete, the cell-activator suspension was added to the RASTRUM™ cartridge. The RASTRUM™ cartridge and 96-well plate were placed into the RASTRUM™ bioprinter. 3D cell-laden structures and hydrogel bridges were printed, and cell suspension was deposited into Geltrex-coated 2D control wells containing Neural Progenitor Medium. After the print process was complete, the hydrogel was checked to ensure gelation, and 200 μL Neural Progenitor Medium was added to the remaining 3D wells. 96-well plates were subsequently placed into the incubator at 37° C. for 3-5 days to allow NPCs to expand. Neural Progenitor Medium was replaced every 48-hours until differentiation.

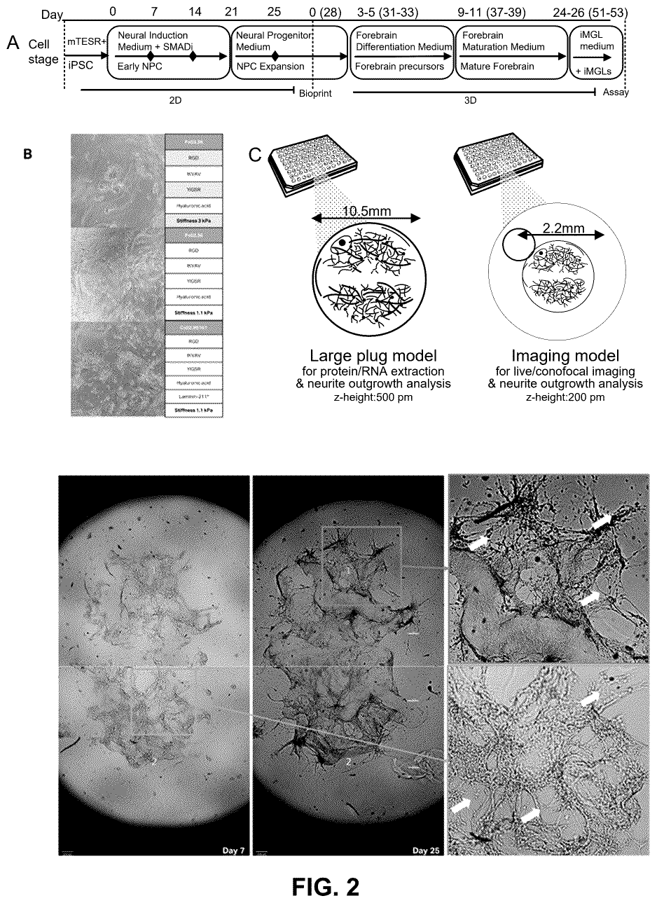

After 3-5 days in Neural Progenitor Medium cells in 2D wells reached confluence, and NPCs within 3D bioprinted constructs had expand to form dense pockets of cells within the outer matrix areas. As with 2D differentiation protocols, Forebrain Neuron Differentiation and Maturation kits from STEMCell Technologies were used to differentiate NPCs. 3-5 days post-printing, media was changed from Neural Progenitor Medium into 200 μL of Forebrain Neuron Differentiation Medium per well. Daily full media changes with Forebrain Neuron Differentiation Medium were performed for 5 days. On Day 6 of forebrain differentiation, media was changed to 200 μL of Forebrain Neuron Maturation medium per well without passage. Cells were maintained in Forebrain Neuron Maturation medium for a further 15 days with half media changes three times per week. After 15 days in Forebrain Neuron Maturation medium, media was changed to 200 μL of iMGL maintenance medium per well (described in 2D differentiation section). iMGLs were thawed and a final concentration of 5% of total cells were added to the 2D and 3D forebrain population cultures. Forebrain cultures with iMGLs were maintained for 48 hours before assay. Live cells were imaged every 8 hours for the first 10 days of culture using brightfield 5× magnification on Incucyte S3 (Sartorius). For an overview culture timeline of 3D model differentiation see FIG. 2A.

Cells were transfected with stable expression Neurolight® third-generation HIV-based VSV-G pseudotyped lentivirus (Sartorius) for the neuron-specific Synapsin promoter which drives expression of red mKate2 in neuronal cell bodies and neurites. Neurolight® was thawed on ice before use. 10 μL per well of Neurolight® Lentivirus was added to a total of 190 μL of Forebrain Differentiation media on Day 1 of forebrain population induction to 3D cultures in 96-well plates. Cells were incubated with the lentivirus for 24 hours before a full media change with 200 μL of Forebrain Differentiation medium on Day 2. Cells were imaged on the Incucyte S3 (Sartorius) and on the INcell analyser (Revvity) at 5× magnification while cells were live and counter-stained with 1×Cell mask green live plasma membrane stain (Thermofisher scientific) following the full culture period.

Cell viability was indicated by the percent of live cells to total cells from Live/Dead assay. Live/Dead staining kit (Thermofisher Scientific) was used to assess cell viability. Assays were performed in bioprinted 96-well plates after full differentiation process was complete. iMGL maintenance media was removed, and the cells were incubated in OptiMEM without phenol red (Gibco) containing 1×NucBlue Live (Thermofisher scientific), 1 mM calcein AM and 2 mM ethidium homodimer I for 30 minutes. Cells were washed with OptiMEM and imaged on the INcell analyser at 20× magnification. The number of total cells were calculated using NucBlue and live (green) cells, and dead (red) cells were counted in each field using Signals Image Artist (SImA) (Revvity). The live/dead cell numbers from five fields and five planes per well were averaged to provide representative results.

Mitochondrial membrane potential was detected with TMRM (tetramethylrhodamine methyl ester) dye. Assays were performed in bioprinted 96-well plates at after 48-hours of iMGL coculture. iMGL maintenance media was removed, and the cells were incubated in OptiMEM without phenol red (Gibco) containing 1×NucBlue Live (Thermofisher scientific), 1×Cell mask green live plasma membrane stain and 200 μM TMRM (Thermofisher scientific) for 30 minutes. Cells were washed with OptiMEM and imaged on the INcell analyser at 20× magnification. The region of interest of cells was determined using Cellmask staining and TMRM intensity per unit area of cell mask staining was calculated per each field using SImA. TMRM intensity was calculated from 5 fields and 5 planes from 12 models per bioprint, for a total of 3 bioprints per condition, these results were averaged to provide representative results.

Reactive oxygen species (ROS) production was detected with DHE (Dihydroethidium) dye. Assays were performed in bioprinted 96-well plates after full differentiation process was complete. iMGL maintenance media was removed, and the cells were incubated in OptiMEM without phenol red (Gibco) containing 1×NucBlue Live (Thermofisher scientific), 1×Cell mask green live plasma membrane stain and 10 μM DHE (Thermofisher scientific) for 30 minutes. Cells were washed with OptiMEM and imaged on the INcell analyser at 20× magnification. The region of interest of cells was determined using Cellmask staining and DHE intensity per unit area of cell mask staining was calculated per each field using SImA. DHE intensity was calculated from 5 fields and 5 planes from 12 models per bioprint, for a total of 3 bioprints per condition, these results were averaged to provide representative results.

Prior to live cell assays (DHE & TMRM), iMGL maintenance media was removed from cells which had been incubated with cocultures for 48 hours. Once media was removed from cells, 50 μL of media samples per well were collected in white bottom 96-well plates and used to measure excreted glutathione (GSH). GSH was detected by adding by adding an equal volume of 300 μM monochlorobimane (MCB) and 100 nM Calcein AM in fluorobrite and incubating at 37 ° C. for 1 hour before reading fluorescence on the Clariostar plate reader (BMG Labtech). Results were calculated from 24 models per bioprint, for a total of 3 bioprints per condition, these results were averaged to provide representative results.

Immunostaining was conducted in situ in 96-well imaging plates (Phenoplate, Revvity) when cell models had reached maturity. 3D models and 2D controls were rinsed with PBS before being fixed with 4% paraformaldehyde for 30 minutes. After rinsing with PBS a further 4 times, cells were permeabilised with 0.1% Triton-X for 20 minutes. Triton-X solution was rinsed with a PBS wash, and 10% donkey serum (NDS) in PBS was added to block for 3 hours at room temperature. Primary antibodies were added in 10% NDS solution and incubated overnight at 4° C. Three PBS washes were conducted, before incubation with secondary antibodies for 2 hours at room temperature. Samples were counterstained with DAPI in mountant (Abcam), and cells were subsequently imaged as detailed in the later microscopy section.

High content imaging was performed using INcell analyser (GE Healthcare) with magnifications 5× and 20× for live cell assays and high content quantification of marker expression following immunofluorescence. Downstream processing of high content images was conducted using SImA (Revvity) to create analysis protocols which were performed using the batch analysis feature. Confocal microscopy was conducted on the Nikon SoRa spinning disk confocal microscope at 40× and 63× magnifications. Downstream analysis for confocal images was conducted using Imaris image analysis software (Oxford Instruments), including 3D rendering, surface rendering, and neurite tracing.

Neurite tracing was performed from βIII-tubulin immunostaining confocal images. Z-stack images of twenty planes spaced at 1 μm were uploaded into Imaris to create a 3D render of neurons. Filament tracer was used to identify neuronal structures. Firstly, cell somas were identified which were over 8 μm in diameter, and branch points from soma were identified using the filament tracer. Neurite identification was improved using the machine learning feature within the software until all neurites were included in the trace, and number of neurites per soma values were extracted from the image details.

Cell ratio of neurons to astrocytes were calculated from immunostaining images for neuronal marker, βIII-tubulin, and astrocyte marker, S100β. High content images were taken of 2D and 3D models in 96-well plate formats at 20× magnification using the INcell analyser. Using randomisation five planes within five fields were selected for the imaging of markers, with a total of twelve wells per condition. SImA software was used to identify cells using DAPI counter stain, and to identify βIII-tubulin+ cells (647nm) and S100β+ cells (488 nm). The number of S100β+ cells were calculated as a percentage of combined βIII-tubulin+ cells and S100β+ cells.

The Mesoscale Discovery Aβ peptide panel 1 kit was used to quantify the ratios of Aβ present in the cultures. Protein was extracted from 2D and 3D model cultures by removing media, rinsing with cold PBS, and incubating with 50 μL of RIPA buffer with 1×protease inhibitors (both Thermofisher scientific) per well. Protein content from samples was quantified using a BCA assay. Mesoscale Discovery Aβ peptide assay was conducted as per manufacturer instructions. In brief, Mesoscale detection plates were blocked with diluent for 1 hour and washed three times before adding 25 μL of detection antibody and 25 μL of samples, controls, and standards (diluted 1:2 and 1:4, three technical replicates per condition) representative results were calculated from 12 models per bioprint, for a total of 3 bioprints per condition. Plates were incubated for a further 2 hours before being washed and read on the Mesoscale instrument. A standard curve was used to calculate the concentration of each isoform within the samples, and results were averaged across dilutions and technical replicates, and were normalised to total protein within the sample.

RNA extraction and bulk RNA-Seq were conducted by Genewiz by Azenta Life Sciences. Samples were prepared for bulk RNA-Seq from 24 models per bioprint, for a total of 3 bioprints per condition. Cell pellets were prepared from 2D samples by dissociating mature cultures with Accutase incubation (8-10 mins, 37° C.), cells were centrifuged at 300×g for 5 mins and frozen at −150° C. Cell pellets were prepared from 3D cultures by incubating with RASTRUM™ fortissimo cell removal solution (Inventia Life Science) for 30 minutes at 37° C. Digested hydrogels and cells were removed from wells and centrifuged at 500×g for 5 mins. Cell pellets were rinsed with PBS to remove remaining hydrogel, and re-centrifuged at 300×g for 5 mins before freezing at −150° C.

RNA-Seq library Preparation, and Sequencing was performed by GeneWiz. RNA samples were quantified using Qubit 4.0 Fluorometer (Life Technologies, Carlsbad, CA, USA) and RNA integrity was checked with RNA Kit on Agilent 5300 Fragment Analyzer (Agilent Technologies, Palo Alto, CA, USA). Invitrogen™ ERCC RNA Spike-In Mix (Cat. No.: 4456740) was used following manufacturer's instructions. RNA sequencing libraries were prepared using the NEBNext Ultra II RNA Library Prep Kit for Illumina following manufacturer's instructions (NEB, Ipswich, MA, USA). Briefly, mRNAs were first enriched with Oligo(dT) beads. Enriched mRNAs were fragmented according to manufacturer's instruction. First strand and second strand cDNAs were subsequently synthesized. cDNA fragments were end repaired and adenylated at 3′ ends, and universal adapters were ligated to cDNA fragments, followed by index addition and library enrichment by limited-cycle PCR. Sequencing libraries were validated using NGS Kit on the Agilent 5300 Fragment Analyzer (Agilent Technologies, Palo Alto, CA, USA), and quantified by using Qubit 4.0 Fluorometer (Invitrogen, Carlsbad, CA). The sequencing libraries were multiplexed and loaded on the flow cell on the Illumina NovaSeq Xplus instrument according to manufacturer's instructions. The samples were sequenced using a 2x150 Pair-End (PE) configuration v1.5. Image analysis and base calling were conducted by the NovaSeq Control Software v1.7 on the NovaSeq instrument. Raw sequence data (.bcl files) generated from Illumina NovaSeq was converted into fastq files and de-multiplexed using Illumina bcl2fastq program version 2.20. One mismatch was allowed for index sequence identification.

The following steps were followed to generate expression matrices from raw sequence reads. We assessed the quality of raw reads (FASTQ format) using FastQC (v 0.12.1). Subsequently, reads were trimmed for residual adaptor sequences using CutAdapt (v 4.6) and their quality reassessed. Post quality control, reads were aligned to the reference genome (GRCh38 Ensembl 108) using the STAR aligner (v 2.7.11a). The aligned reads were quantified using featureCounts (Subread package v 2.0.6) to generate a count matrix. A custom script was used to produce a TPM matrix from gene counts and their effective length.

Sample quality control (QC) was conducted using the TPM matrix to examine the effects of batch and outlier samples. We adopted two methods, PCA analysis and sample-to-sample similarity matrices for this evaluation. PCA analysis was performed on the TPM matrix using the FactoMineR package. The resultant principal components were examined to assess batch effects. The sample-to-sample similarity matrix was generated by calculating pairwise Pearson correlations across all samples based on their TPM based transcriptomic profiles. The resultant matrix was visualized using a heatmap and clustered using Ward's hierarchical clustering to examine sample groupings.

To identify differentially expressed genes (DEG), count matrices were used for selected sample groups while accounting for batch effects in the DESeq2 package. Genes were considered significantly up/downregulated if they exhibited an extreme fold change (greater than 1.2 times), a significant adjusted P-value (less than 0.05), and minimal normalized expression values across samples (baseMean value greater than 10). Pathway enrichment analysis was conducted using the clusterProfiler package and four databases: GO, KEGG, MsigDB, and Reactome. Only significantly enriched pathways (adjusted P-value <0.05) were considered for functional annotation and interpretation. The results provided insight into the biological processes underlying the expression data. To visualize the overrepresentation of specific pathways, lollipop plots were produced from the top ten enriched terms for each condition and direction.

All data are expressed as mean+/−SEM unless otherwise stated. Graphs and statistical analysis were made and performed in GraphPad Prism. Statistical tests were always performed using two-way ANOVA with Tukey's post hoc test unless otherwise indicated. A P-value of <0.05 was considered statistically significant. For all graphs displaying statistical analysis, results represent the average of at least 12 models per bioprint, across three replicate bioprints, each containing NPCs generated from the same batch of dual SMAD inhibition differentiation.

### Results

Forebrain cortical differentiation protocols were first optimized in 2D before transitioning to 3D systems. Healthy iPSCs were differentiated into NPCs using dual SMAD inhibition, a process that activates neural fate acquisition through inhibition of the BMP and TGF-β pathways. The resulting NPCs expressed hallmark progenitor markers SOX1 and SOX2, demonstrating ectodermal lineage specification. Further differentiation into forebrain cortical neurons produced mixed populations of excitatory and inhibitory neurons alongside astrocytes, which expressed synaptic proteins.

The translation of these differentiation protocols into a 3D platform involved careful selection of a hydrogel scaffold that could support NPC proliferation and differentiation. An initial matrix screening was performed to test three candidate hydrogels with varying stiffness and biochemical compositions: Px03.36 (3 kPa, peptides+hyaluronic acid (HA)), Px02.36 (1.1 kPa, peptides+HA), and Cx02.99161 (1.1 kPa, peptides+HA+laminin-211). Limited migration and expansion were observed in Px03.36 and Px02.36, however, the addition of laminin-211 in the Cx02.99161 matrix significantly enhanced cell clustering and neurite outgrowth, highlighting its suitability as a matrix for the forebrain culture (FIG. 2D). Laminin is known to play a key role in neuronal adhesion and synapse formation, with the laminin-211 isotype being particularly abundant in the cortex, thus explaining the improved NPC outcomes in Cx02.99161.

Building on these results, a tri-matrix system was developed to allow precise tracking of neurite outgrowth across a hydrogel bridge. In this model, NPCs were bioprinted into two distinct cell populations within matrix Cx02.99161, connected by a bridging hydrogel area made of Px03.36 (3 kPa). This design aimed to delay soma migration while facilitating neurite projection, creating a controlled environment to study neural connectivity (FIG. 2C). By Day 7 post-print, cell migration into the bridging zone was restricted, but smaller neurite extensions were populating the area, demonstrating the effectiveness of the matrix in spatially constraining cell soma. By Day 25 post-print, neurite outgrowth successfully bridged the two cell populations, establishing robust connectivity (FIG. 2A).

Two distinct tri-matrix model designs were created for specific applications. The “large plug model,” spans the width of a 96-well plate and measures 500 μm in Z-height. This model conformation was configured for downstream analyses such as RNA extraction or biochemical assays (FIG. 2C, Large Plug Model). Meanwhile, a smaller imaging model (220 μm width, 200 μm Z-height) was designed for high-resolution confocal imaging, enabling more detailed visualisation of neurite processes and synaptic structures (FIG. 2C, Imaging Model). Both designs supported the establishment of neuronal networks, with neurites extending across the hydrogel bridge.

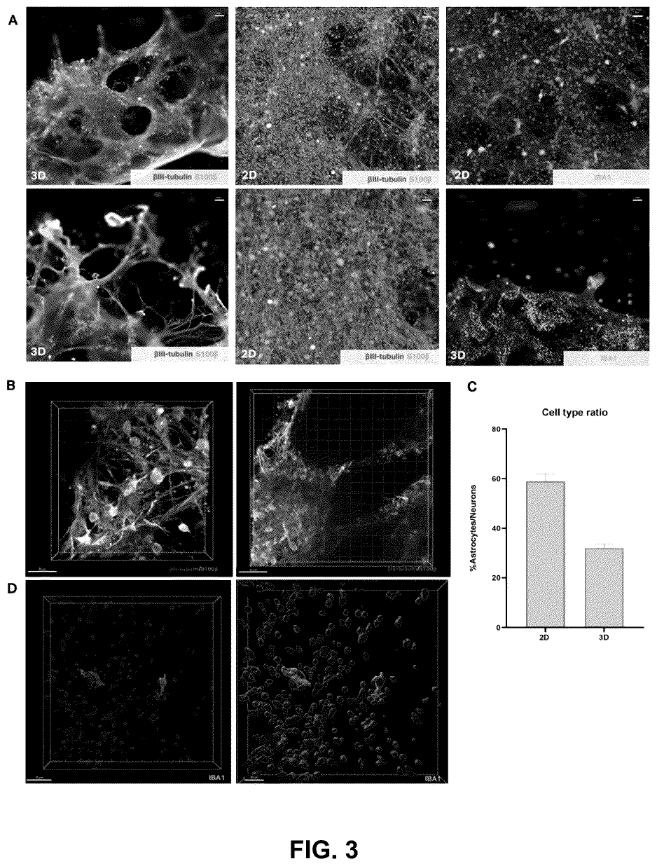

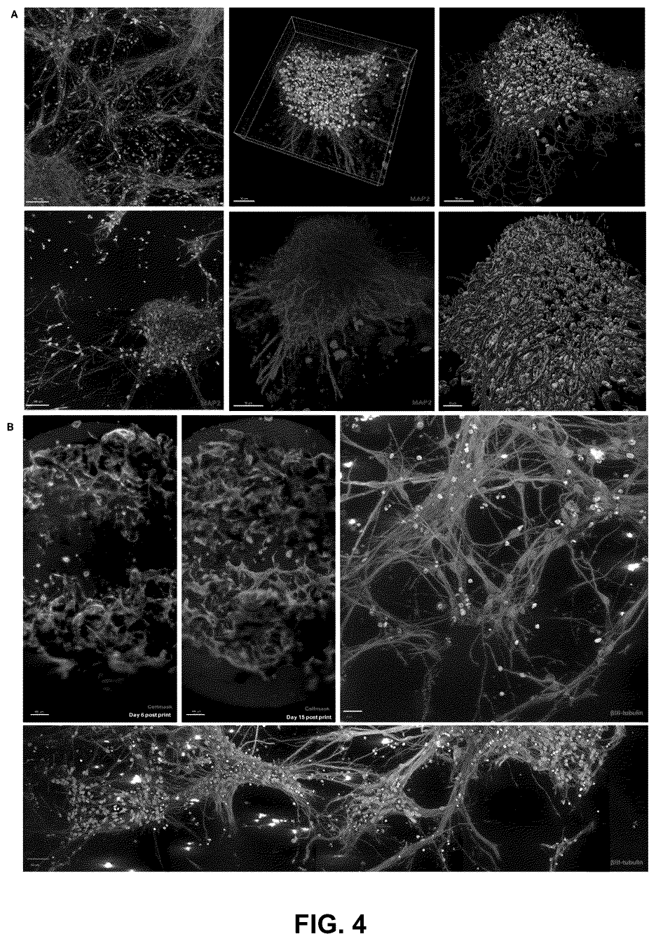

Immunostaining revealed distinct differences in cell composition and morphology between 2D and 3D cultures. Astrocytes, marked by S 100β expression, formed monolayers underneath neurons in 2D cultures and comprised approximately 60% of the total population (FIG. 3A). In contrast, in 3D cultures, astrocytes were distributed within cell clusters and formed extended projections, representing about 30% of the total population (FIGS. 3C & 3D). Induced microglial-like cells (iMGLs) were introduced in the final days of culture, with IBA1 staining confirming their morphology and distribution amongst other cell types within the 3D scaffold (FIG. 3B). Mature neurons in 3D cultures expressed the neuronal markers βIII-tubulin and MAP2, and confocal imaging of cells within the hydrogel bridge shows that their neurites are forming networks (FIGS. 4A & 4B). 3D rendering and filament tracing of these confocal images allows neurite networks to be visualised, providing a comprehensive view of neuronal connectivity (FIG. 4A).

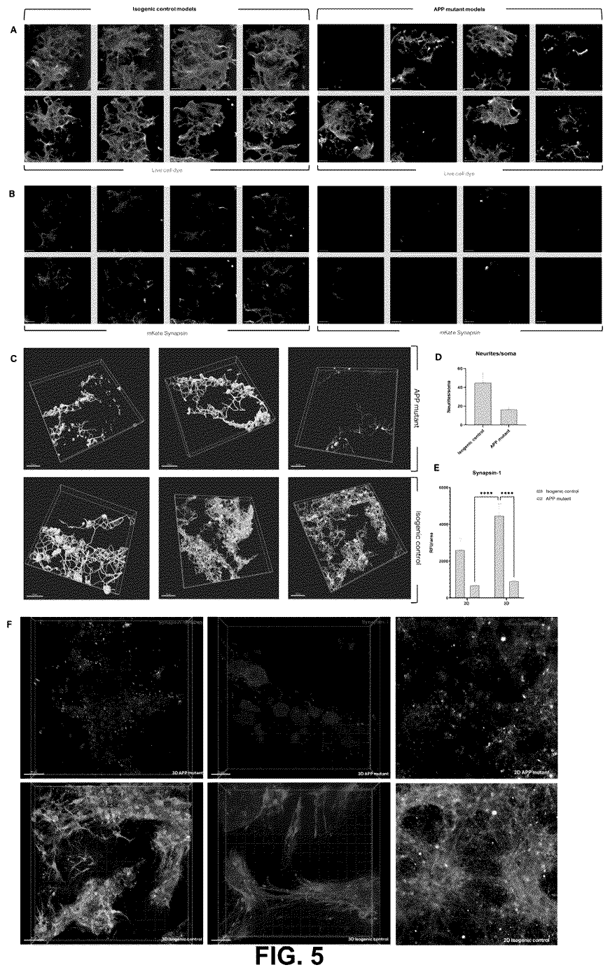

The tri-matrix model was also used to study neural network formation in iPSCs carrying APP mutations to model AD in 2D and 3D conditions. These models were also compared to their iso-genic controls across assays. Healthy controls demonstrated robust neurite outgrowth across the hydrogel bridge, with neurite bundles connecting cell clusters (FIGS. 2A & 4B). In contrast, APP mutant cultures exhibited impaired neurite connectivity and reduced cluster formation at the NPC stage, suggesting an early developmental defect (FIG. 5A). Synaptic deficits, a hallmark of AD, were also observed in APP mutant cultures. Transfection with mKate-tagged synapsin revealed reduced synapsin expression in APP mutant neurons compared to controls, indicating disrupted synaptogenesis (FIG. 5B). Viability assays confirmed significantly lower cell viability in APP mutants, consistent with their impaired connectivity.

Confocal imaging for βIII-tubulin staining was used to render neurite connectivity traces (FIG. 5C). Healthy controls exhibited extensive neurite networks, while APP mutants displayed fewer, fragmented neurites with visible debris, indicating increased degeneration. Metrics from this filament tracing analysis confirmed a significant reduction in neurites per soma in APP mutants (FIG. 5D). Immunostaining for synaptic proteins such as Synapsin-1 and PSD95 revealed significantly reduced expression in APP mutants in 3D cultures, with a less pronounced difference in 2D (FIGS. 5E & 5F). These results underscore the enhanced sensitivity of 3D models in detecting subtle synaptic deficits associated with APP mutations.

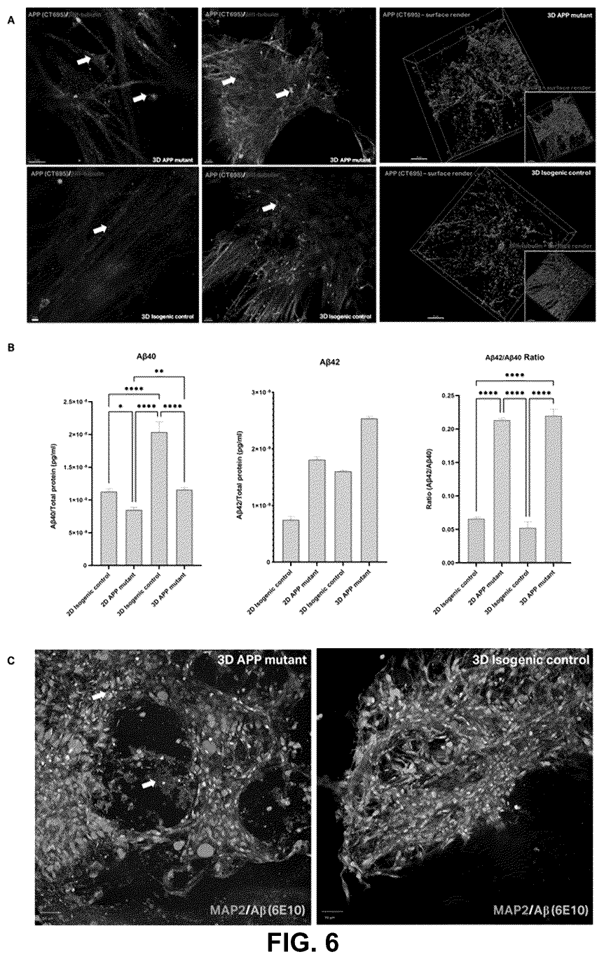

The pathological consequences of APP mutations, including altered APP processing and Aβ production, were assessed in both 2D and 3D cultures. APP C-terminal fragments (CTFs) have been implicated in mitochondrial dysfunction and proteostasis deficit, were shown to be more broadly distributed in APP mutants in 3D compared to isogenic controls (FIG. 6A). This suggests that the 3D environment may exacerbate proteostasis challenges, highlighting its relevance for studying AD pathology. Aβ levels (Aβ40 and Aβ42) were measured using mesoscale analysis, revealing changes to the Aβ40/42 ratio in 2D and 3D cultures (FIG. 6B). Immunostaining also confirmed the localization of Aβ around cells and within the hydrogel in APP mutant 3D cultures (FIG. 6C).

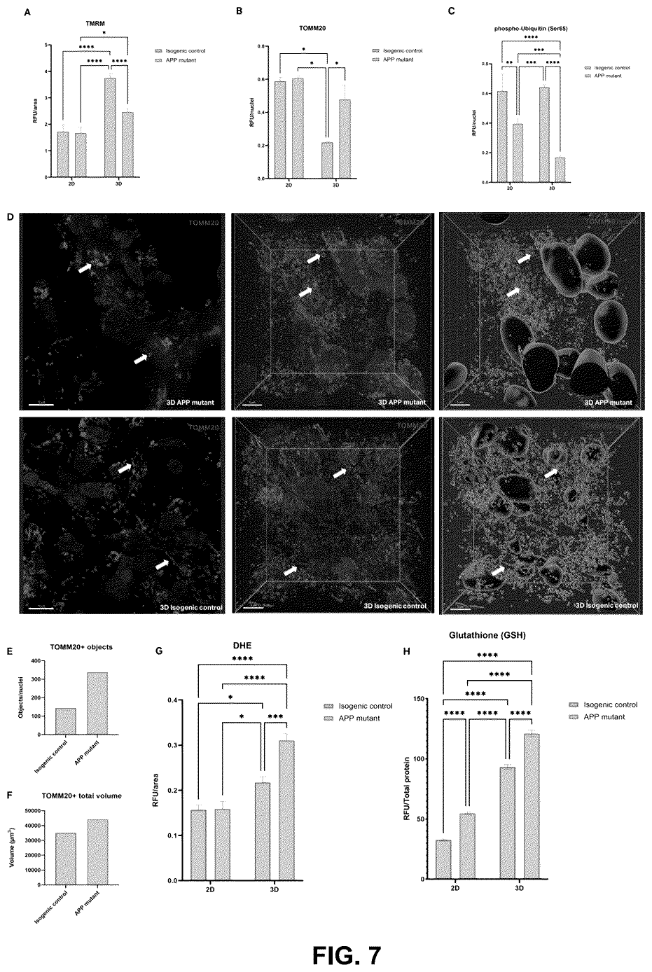

Swedish and Indiana APP mutations have been widely suggested to result in mitochondrial dysfunction and increased production of ROS. It has been suggested that poor processing of APP and the resultant changes to amyloid processing pathways causes mitochondrial stress and damage to mtDNA, driving cellular energy imbalances. The dysfunctional mitochondria are not broken down by mitophagy, and this results in oxidative stress. To determine the extent of mitochondrial dysfunction within the models, live cell TMRM assays were used. TMRM is fluorescent when accumulating in mitochondria with an active membrane potential, and thus, an increase in TMRM signal is indicative of increased mitochondrial health. In FIG. 7A, there is no significant difference between TMRM fluorescence in isogenic control and APP mutant cultures in 2D. However, in 3D, there is greater fluorescence in the 3D isogenic control compared to the 2D isogenic control. Furthermore, in the 3D cultures, TMRM fluorescence is reduced in APP mutant compared to the isogenic control.

Mitochondrial function was further investigated with immunofluorescent staining for TOMM20, a mitochondrial outer membrane protein, which can give an indication of mitochondrial number. Quantification of TOMM20 staining shows no change between isogenic control and APP mutant cultures in 2D (FIG. 7B). However, in 3D cultures, the isogenic control cultures have significantly less TOMM20 expression in comparison to 2D. Furthermore, TOMM20 staining also increases in the APP mutant 3D cultures. An increase in TOMM20 staining could suggest an increased number of mitochondria, however, as TMRM staining is inversely correlated to TOMM20, this indicates that the increased number of mitochondria have a lower membrane potential and therefore are likely to be dysfunctional. Taken together, these results suggest a mitophagy deficit. To investigate this further, cultures were immunostained for phospho-Ubiquitin (pUb). In both 2D and 3D cultures, the APP mutant cells show a decrease in pUb, with the 3D samples showing a more exaggerated change (FIG. 7C). This supports the hypothesis of dysfunctional mitophagy in the APP mutant cultures.

To visualize the morphology of the mitochondria in the 3D cultures, TOMM20 was imaged using confocal microscopy. TOMM20 staining shows elongated chains of mitochondria (FIG. 7D, indicated by white arrows) in the isogenic control 3D cultures, whereas in the APP mutant 3D cultures, mitochondria appear rounded and fragmented (FIG. 7D, indicated by white arrows). TOMM20 and DAPI staining were rendered as surfaces to quantify mitochondrial morphology features, as shown in FIG. 7D, the total number of objects and total volume were increased in 3D APP mutants (FIGS. 7E & 7F).

An accumulation of dysfunctional mitochondria which are not being cleared by mitophagy can be associated with an increase in oxidative stress. Live DHE (Dihydroethidium) assays were used to investigate the production of ROS within the cultures. DHE is a fluorescent probe for the detection of ROS, specific for superoxide and hydrogen peroxide. As shown in FIG. 7G, no difference can be observed between DHE intensity in 2D isogenic control and APP mutant cultures, however, 3D healthy isogenic control samples have higher baseline levels of ROS production. Furthermore, 3D cultures of APP mutant cells have further increased production of ROS.

Glutathione (GSH) is an important cellular antioxidant thus the production of GSH from 2D and 3D cultures was measured from media samples. As shown in FIG. 7H, 2D cultures have lower levels of secreted GSH than 3D cultures in both isogenic control and APP mutant cells. Additionally, in both culture conditions, APP cells have increased secretion of GSH into the media. In the 3D culture condition, GSH production correlates with increased production of ROS, indicating that antioxidant production may be upregulated in response to increased cell stress.

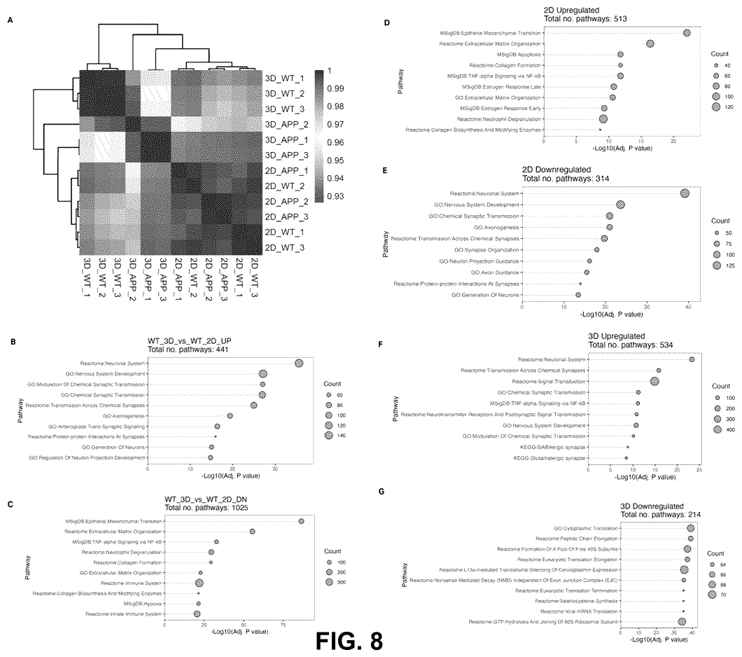

To further investigate the changes between the 2D and 3D cultures of both isogenic and APP mutant cell cultures, cells were removed from within the hydrogel or from the culture plate using enzymatic digestion and pelleted for RNA-Seq from three bioprints per condition. Quality control analysis determining the relationship between samples demonstrates low batch-to-batch variability, with samples within each sample group clustering together regardless of their batch (FIG. 8A). Sample groups were compared based on the similarity of their transcriptomic profile (FIG. 8A). The heatmap of sample similarity shows that all 2D cultures including isogenic controls, and APP mutants, have a high degree of similarity, thereby grouping together. In contrast, 3D cultures of isogenic control and APP mutant samples group independently, indicative of a lower similarity between these sample groups. In addition, 3D isogenic control cultures show a low degree of similarity to the 2D isogenic control cultures as they group independently.

Differential gene expression (DEG) analysis was conducted to compare (1) 3D vs 2D isogenic controls; (2) 3D APP mutant vs 3D isogenic control; and (3) 2D APP mutant and isogenic control. The number of significant upregulated and downregulated DEGs were similar between comparisons ranging from ~2,800 to 2,200 genes. To understand the biology behind these differences, pathway analysis was conducted on DEG for each comparison (FIGS. 8B-G). On comparing 3D and 2D isogenic controls, upregulated genes in 3D cultures were found in pathways involved in nervous system development, axonogenesis, generation of neurons and synaptic transmission, indicating that 3D cultures are more mature compared to their 2D counterparts. Downregulated genes in 3D cultures compared to 2D cultures of isogenic control cells (FIG. 8C) include genes associated with pathways for immune signalling, hypoxia, and extracellular matrix organisation. This indicates that cells in 3D cultures have a less reactive phenotype than 2D cultures, and that being embedded within hydrogel does not induce hypoxia within the cultures. Downregulation of extracellular matrix reorganisation associated pathways in 3D cultures could be due to the cells in 3D conditions having retained deposited ECM proteins within the hydrogel, unlike 2D cultures, where ECM proteins will be rinsed off during media changes.

Comparing 2D APP mutants to 2D isogenic control cultures, upregulated genes (FIG. 8D) are associated with pathways for immune signalling, cytokine activity, apoptosis, and collagen synthesis. Whereas downregulated genes (FIG. 8E) are associated with pathways for nervous system development, synaptic transmission, and axonogenesis. This demonstrates that in 2D cultures, APP mutations are disrupting neuronal growth and synaptic transmission, and making cells take on a more reactive phenotype. Conversely, when comparing 3D APP mutants with 3D isogenic control cultures upregulated genes (FIG. 8F) are significantly enriched in pathways for signal transduction, synaptic transmission, and neurotransmitters. This is at odds with protein analysis work which demonstrated that synaptic proteins are significantly less expressed in the APP mutant cultures, and that neuronal outgrowth was significantly reduced. However, the downregulated genes in 3D APP mutant vs 3D isogenic control cultures (FIG. 8G) are associated with pathways for translation of proteins and ribosomal function. This suggests that although genes are upregulated, they are not being translated into functional proteins.

In summary, it is evident that there are significantly different changes to cellular mechanisms and protein expression between 2D and 3D cultures, due to the culture conditions of the cells (2D vs 3D), and this is further exaggerated by the disparate changes between the APP mutants in 2D and 3D culture conditions.

Overall, these findings highlight the advantages of using 3D culture systems for modelling neurodevelopmental and neurodegenerative diseases. The tri-matrix model not only recapitulates key aspects of neural connectivity and synaptic organization but also amplifies disease-relevant phenotypes, providing a powerful platform for studying AD and other disorders.

### Analysis

As demonstrated above, a tri-matrix 3D bioprinted scaffold-based model can be applied assess neurite outgrowth and neural connectivity using human iPSC-derived NPCs differentiated into fore-brain cortical cocultures. The model was validated in the context of AD using APP mutant NPCs, recapitulating critical features of neurodegenerative pathology. The findings demonstrate that the tri-matrix 3D system provides a physiologically relevant environment for studying neural interactions and connectivity, while addressing the limitations of conventional 2D cultures. The APP mutant model revealed significant reductions in neurite outgrowth, synaptic protein expression, and matrix remodeling, alongside underlying mitochondrial dysfunction, oxidative stress, and proteostasis disruption. Many of these pathological features were not observed robustly in the corresponding 2D cultures, emphasizing the value of 3D systems to capture disease-relevant phenotypes.

Considerable progress has been made in the development of neural 3D models, but few systems can be implemented in the quantitative assessment of neurite outgrowth and connectivity. Our tri-matrix 3D bioprinted model addresses two major limitations in this field: optical clarity for high-resolution real time imaging and compatibility with medium-throughput analysis in 96-well plate formats.

Traditional models, such as neural organoids and neurospheroids offer insights into brain-like structures but present practical challenges for application. Neural organoids are limited by their labour-intensive setup process and low throughput format, while neurospheroids and assembloids, although generally more scalable, often have dense, intertwined neuronal structures that obscure individual neurite resolution. The hydrogel bridge in the tri-matrix model, designed with increased stiffness (3 kPa) to restrict soma migration while permitting neurite extension, creates an optically clear environment where neurite projections can be easily imaged and quantified.

The increased hydrogel stiffness in the central matrix, achieved through higher crosslinking density reduces pore size, initially prevents large cell soma from populating the hydrogel bridge while still allowing neurite penetration. This ensures that the central region remains sparsely populated, enabling high-resolution imaging and quantitative analyses of neurite growth metrics, such as neurite length, branching complexity, and neurites per soma. Additionally, the total printing time of 32 minutes per 96-well plate improves model practicality for medium-throughput applications, facilitating its potential use in industrial applications.

While the 28-day culture duration may pose a challenge for high-throughput applications compared to simple 2D assays, this culture time remains significantly shorter than standard organoid protocols, which often require around 100 days for full maturation, while offering the same benefit of a self-assembling complex model system. Additionally, within the 28-day period, transcriptomic analysis reveals that the 3D system can achieve a higher degree of neuronal maturation and synaptic connectivity within the 28 days, indicating that this model provides a more translatable model within a shorter time frame.

The tri-matrix model was adapted to create a model of AD to demonstrate translatability and application of the system in disease modelling, using APP mutant NPCs. In 3D culture conditions, neurite outgrowth across the hydrogel bridge was significantly reduced in APP mutant cells compared to isogenic controls. APP mutant neurites were fragmented, shorter, and exhibited reduced branching complexity, alongside a marked reduction in synapsin-1 expression, indicating impaired synapse formation. In contrast, APP mutant cells cultured under 2D conditions displayed little difference in morphology or synaptic protein expression compared to controls. These findings highlight the ability of the 3D bioprinted system to capture early disease pheno-types that remain undetected in traditional 2D cultures.

Transcriptomic analysis demonstrated greater neuronal maturation in 3D cultures compared to 2D cultures in isogenic controls. Genes related to axonogenesis, synaptic transmission, and nervous system development were upregulated, indicating that 3D environments promote enhanced neuronal differentiation and maturation. However, transcriptomic analysis also showed distinct molecular mechanisms implicated in neurite deficits between the 2D and 3D APP mu-tant cultures. In 2D conditions, downregulation of genes associated with neuronal growth and synaptic transmission was observed. Conversely, in 3D APP mutant cultures, pathways related to protein translation and ribosomal function were downregulated, consistent with impaired protein synthesis. The downregulation and reduced expression of ribosomal proteins and translation machinery has been previously described in APP models. Additionally, the relationship between APP and synaptogenesis is complex, with evidence for APP mutations resulting in a synaptogenic effect in some culture conditions. These results show that the reduced expression of ribosomal proteins could be seen to result in failure to translate the increased synapse-related genes into functional proteins, which would result in the observed deficits in neurite connectivity and synapse formation.

To understand the cellular mechanisms contributing to neurite outgrowth changes between 2D and 3D APP mutant cultures, we examined mitochondrial function, oxidative stress, and proteo-stasis, which are well-established hallmarks of AD. APP mutant cultures displayed disrupted Aβ processing, evidenced by altered Aβ40/42 ratios and increased APP C-terminal fragment and Aβ accumulation in the hydrogel. However, significant mitochondrial dysfunction and oxidative stress were only observed in 3D cultures.

Mitochondria play a central role in neurite outgrowth by supplying ATP and calcium buffering to support cytoskeletal remodelling and local energy demands. In 3D APP mutant cultures, we observed reduced mitochondrial membrane potential and fragmented mitochondrial networks. Notably, baseline mitochondrial activity in isogenic 3D cultures was also higher than in 2D cultures, consistent with previous reports of increased energy demands in 3D environments. This elevated baseline energy requirement may exacerbate mitochondrial dysfunction in disease contexts, limiting the availability of ATP for neurite extension and maintenance.

Oxidative stress also contributes to cellular damage and mitochondrial dysfunction. APP mutant 3D cultures exhibited elevated ROS levels compared to controls, alongside increased GSH production, indicating a compensatory antioxidant response. Despite this, production of ROS persisted, likely creating a feedforward cycle of mitochondrial damage, energy deficits, and im-paired neurite outgrowth.

Additionally, APP mutant 3D cultures displayed impaired proteostasis, as evidenced by reduced pUb levels. The accumulation of misfolded proteins, including Aβ, further increases cellular stress and compromises mitochondrial function. Together, the combination of mitochondrial dysfunction, production of ROS, and proteostasis disruption in APP mutant 3D cultures provides a mechanistic explanation for the pronounced neurite deficits observed in this system.

The 3D bioprinted model represents a significant advancement. Further advancements possible using the overall framework described above include using the model for patient derived cells to fully capture pathology in sporadic AD patients. Additionally, while bulk RNA sequencing provides valuable insights into gene expression changes, single-cell RNA sequencing can offer a more detailed understanding of cellular heterogeneity within 3D cultures. Additionally, pharmacokinetic analysis may be incorporated to assess drug distribution, metabolism, and clearance within the 3D system, offering further insights into compound bioavailability and efficacy to provide a robust view of translatability. Finally, the scalability of the 3D bioprinted model for true high-throughput screening applications of thousands of compounds can be further optimized, such as by expanding beyond 96-well plate implementations to larger batches.

The field of advanced translational in-vitro models remains in its infancy, and in this light, in vivo models of AD remain valuable. In vivo models of AD allow for the study of systemic interactions, neuroimmune responses, and behavioral phenotypes that cannot yet be replicated in vitro. These models provide insights into the progression of AD within a living organism, also allowing for the study of blood-brain barrier dynamics, neurovascular interactions, and long-term pathological changes over time. Advanced 3D models, such as the disclosed tri-matrix bioprinted model, offer a complementary approach by enabling tightly controlled studies of human-specific neuronal interactions, synaptic dysfunction, and disease phenotypes at the cellular level. By integrating 3D models alongside in vivo studies, it is possible for researchers to refine experimental design, reducing the number of animals required while improving mechanistic understanding before proceeding to whole-organism studies.

Conventional 2D models for studying AD have significant limitations, which underscore the value of 3D bioprinted models in capturing the complexity of disease pathology. The 3D environment not only promotes neuronal maturation but also amplifies key features of AD, including synaptic dysfunction, mitochondrial impairment, and oxidative stress. This makes 3D bioprinted models a powerful tool for studying the cellular mechanisms underlying AD and for screening potential therapeutic interventions. The ability to track neurite outgrowth and connectivity in real time using the hydrogel bridge within this triple-matrix model provides a unique advantage and potential assay output for future studies of neurodegeneration, and as a template model for other neurological diseases

### Additional Considerations

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate +/−10% unless another meaning is apparent from the context. For example, “approximately ten” should be understood to mean “in a range from nine to eleven.”

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs. Thus, while particular embodiments and applications have been illustrated and described, it is to be understood that the described subject matter is not limited to the precise construction and components disclosed. The scope of protection should be limited only by the following claims.

What is claimed is:

1. A three-dimensional (3D) bioprinted model for neurite outgrowth, the 3D bioprinted model comprising:
a first cell-laden compartment comprising a first population of neural cells embedded in a first hydrogel matrix;
a second cell-laden compartment comprising a second population of neural cells embedded in the first hydrogel matrix; and
an optically-clear hydrogel bridge positioned between and connecting the first and second hydrogel matrices, the optically-clear hydrogel bridge comprising a second hydrogel matrix that restricts soma migration while permitting neurite extension across the optically-clear hydrogel bridge.

2. The 3D bioprinted model of claim 1, wherein the first hydrogel matrix has a first stiffness and the second hydrogel matrix has a second stiffness, greater than the first stiffness.

3. The 3D bioprinted model of claim 2, wherein the first stiffness is approximately 1.1 kPa and the second stiffness is approximately 3 kPa.

4. The 3D bioprinted model of claim 1, wherein the first hydrogel matrix comprises peptides, hyaluronic acid, and laminin-21.

5. The 3D bioprinted model of claim 1, wherein the second hydrogel matrix comprises peptides and hyaluronic acid.

6. The 3D bioprinted model of claim 1, wherein the 3D bioprinted model fits within a well of a 96-well plate.

7. The 3D bioprinted model of claim 1, wherein the hydrogel bridge has a width separating the cell-laden compartments of approximately 1 mm to approximately 5 mm.

8. The 3D bioprinted model of claim 1, wherein the model has a diameter of approximately 2.2 mm to approximately 10.5 mm and a Z-height of approximately 200 μm to approximately 500 μm.

9. The 3D bioprinted model of claim 1, wherein at least one of the first population of neural cells or the second population of neural cells comprises neural progenitor cells.

10. The 3D bioprinted model of claim 9, wherein the neural progenitor cells comprise glutamatergic neurons, GABAergic neurons, astrocytes, and microglia.

11. The 3D bioprinted model of claim 9, wherein the neural progenitor cells are derived from human induced pluripotent stem cells.

12. The 3D bioprinted model of claim 11, wherein the human induced pluripotent stem cells carry amyloid precursor protein mutations.

13. The 3D bioprinted model of claim 12, wherein the amyloid precursor protein mutations comprise K670M/N671L and V717F mutations.

14. A method for quantifying neurite outgrowth, the method comprising:
providing a 3D bioprinted model comprising:
a first cell-laden compartment comprising a first population of neural cells embedded in a first hydrogel matrix;
a second cell-laden compartment comprising a second population of neural cells embedded in the first hydrogel matrix; and
an optically-clear hydrogel bridge positioned between and connecting the first and second hydrogel matrices, the optically-clear hydrogel bridge comprising a second hydrogel matrix that restricts soma migration while permitting neurite extension across the optically-clear hydrogel bridge;
culturing the neural cells such that neurites extend from the first and second cell-laden compartments into the optically-clear hydrogel bridge; and
quantifying neurite outgrowth across the optically-clear hydrogel bridge based on imaging of the optically-clear hydrogel bridge.

15. The method of claim 14, wherein the imaging comprises confocal microscopy imaging of the hydrogel bridge.

16. The method of claim 15, wherein the quantifying comprises performing filament tracing analysis on confocal microscopy images to determine at least one of neurite length, branching complexity, or number of neurites per soma.

17. The method of claim 14, wherein the neural cells comprise neural progenitor cells are derived from human induced pluripotent stem cells that carry amyloid precursor protein mutations.

18. The method of claim 17, wherein the amyloid precursor protein mutations comprise K670M/N671L and V717F mutations.

19. The method of claim 14, further comprising screening a therapeutic compound for efficacy in treating a disease based on the quantifying of the neurite outgrowth.

20. The method of claim 19, wherein the disease is Alzheimer's disease.
